SEQUENCE LISTING (2 sequences)

SEQ ID NO: 1 - protein, 1971 aa, synthetic construct
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHD

SEQ ID NO: 2 - protein, 1351 aa, synthetic construct
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVR

(19) United States
(12) Patent Application Publication
(10) Pub. No.: US 2026/0265294 A1
(43) Pub. Date: Sep. 10, 2026

(54) PURIFICATION OF SOLUBLE COMPLEMENT RECEPTOR AND VARIANTS THEREOF

(71) Applicant: CSL Innovation Pty Ltd., Melbourne, Victoria (AU)

(72) Inventors: Erik HINZE, Marburg (DE); Joerg LEMMER, Ebsdorfergrund (DE); Olga MUELLER, Marburg (DE); Irina KOKKINOS, Linden (DE)

(21) Appl. No.: 18/875,488

(22) PCT Filed: Jun. 16, 2023
(86) PCT No.: PCT/AU2023/050538

§ 371 Date: Dec. 16, 2024

(30) Foreign Application Priority Data
Jun. 17, 2022 (EP) 22179637.8

(51) Int. Cl. C07K 1/20; B01D 15/32; B01D 15/36; C07K 1/18; C07K 14/705

(52) U.S. Cl. CPC C07K 1/20; B01D 15/327; B01D 15/362; B01D 15/363; C07K 1/18; C07K 14/70596; C07K 2319/21

(57) ABSTRACT

The present invention pertains to the purification of soluble complement receptor proteins and variants thereof by hydrophobic interaction chromatography (HIC) using a chromatographic material with a particle size of less than 60 μm.

### RELATED APPLICATION DATA

The present application claims priority from European Patent Application No. 22179637.8 entitled ‘Purification of soluble complement receptor and variants thereof’ filed 17 Jun. 2022, the entire contents of which is hereby incorporated by reference.

### SEQUENCE LISTING

The present application is filed together with a Sequence Listing in electronic format. The entire contents of the Sequence Listing are hereby incorporated by reference.

### FIELD OF THE INVENTION

This invention relates to the purification of complement receptor proteins and variants thereof. The invention provides a hydrophobic interaction chromatography (HIC) step allowing purification of soluble complement receptor and variants thereof at high yield, in particular of soluble complement receptor type 1 and variants thereof.

### BACKGROUND OF THE INVENTION

Protein purification from a mixture can be based on differences in the molecular properties such as size, charge and solubility. Corresponding protocols are called size exclusion chromatography, ion exchange chromatography, differential precipitation and the like. The chromatographic principle is typically based on a system with at least two phases, a stationary phase and a mobile phase, wherein the mobile phase is running through the stationary phase. A mixture comprising the target protein is solved in a mobile phase. The stationary phase interacts with the target protein in form of non-covalent binding. This interaction allows the target protein to be held back, while the residual mobile phase flows through the stationary phase. The target protein can then be eluted using a suitable elution buffer.

HIC separates molecules on the basis of their surface polarity. The chromatographic materials display nonpolar groups (hydrophobic ligands) on their surfaces which interact with the hydrophobic surface area of a given molecule. The strength of the hydrophobic interaction depends inter alia on the level of hydrophobicity of the chromatographic material, i.e. the density of the hydrophobic ligand, pH, solvent and its ionic strength.

The complement system is part of the innate immune system and involves a number of cell-surface and soluble proteins that play a role in elimination of foreign microorganisms, whilst protecting the host from complement-related damage. The complement system comprises soluble components C1-C9 and becomes activated when its primary components are fragmented and the fragments activate additional complement proteins resulting in a proteolytic cascade. Activation of the complement system leads to increased vascular permeability, chemotaxis of phagocytic cells, activation of inflammatory cells, direct killing of cells and tissue damage.

Complement receptor type 1 (CR1) is a principal regulator of the activation of complement. CR1 is present on the membranes of erythrocytes, monocytes/macrophages, granulocytes, B cells, some T cells, splenic follicular dendritic cells, and glomerular podocytes. CR1 binds to C3b and C4b and is referred to as the C3b/C4b receptor, as it functions as a negative regulator of C3 activation and can inhibit each of the classical, lecithin and alternative pathways.

The primary sequence of CR1 has been determined (Klickstein et al., J. Exp. Med. 165:1095-1112 (1987), Klickstein et al., J. Exp. Med. 168:1699-1717 (1988); Hourcade et al., J. Exp Med. 168:1255-1270 (1988)). It is composed of 30 short consensus repeats containing 60-70 amino acids, of which 29 are conserved. A naturally occurring soluble form of CR1 (sCR1) has been detected in the plasma of normal individuals and certain individuals with Systemic lupus erythematosus (SLE) (Yoon & Fearon J. Immunol. 134:3332-3338 (1985)).

Recently, a human sCR1 variant, truncated at amino acid 1392 was found to retain the complement regulatory activity of the full-length protein being a potent inhibitor of complement activation. The fragment, called CSL040, shows affinity to C3b and C4b as well as its cleavage and decay acceleration activity and was able to prevent organ damage in a glomerulonephritis model through a reduction in cellular infiltrates and urine albumin. CSL040 has a modular structure comprising three long homologous repeat domains, LHR-A, LHR-B and LHR-C, but not LHR-D. CSL040 was thus reported as a candidate for treatment of complement-mediated disorders (Wymann, S., Dai, Y., Nair, A. G., Cao, H., Powers, G. A., Schnell, A., . . . & Hardy, M. P. (2021). A novel soluble complement receptor 1 fragment with enhanced therapeutic potential. Journal of Biological Chemistry, 296). Aspects of CSL040 and further sCR1 variants are described in WO2019/218009.

Known purification protocols give insufficient yield and/or purity and thus require further purification steps. Hence, there is a need for improved methods for purifying complement receptor proteins and variants thereof.

### SUMMARY OF THE INVENTION

The inventors found that the yield of soluble complement receptor using HIC can be improved with a particulate HIC material comprising agarose and a hydrophobic ligand having a low particle size. The invention therefore relates to the methods defined in the following items and further described herein.

Item 1: A method for purifying a soluble complement receptor protein (sCR) or a variant thereof, in particular CR1 or a variant thereof, comprising subjecting a liquid comprising said complement receptor protein or variant thereof to a hydrophobic interaction chromatography (HIC), wherein said HIC comprises applying said liquid to a particulate HIC material comprising agarose and a hydrophobic ligand, wherein the particle size of said particulate HIC material is less than 60 μm.

Item 2: The method of item 1, wherein the particle size is less than 55 μM, preferably less than 50 μM.

Item 3: The method of item 1, wherein the particle size is from 20 μm to 55 μm, in particular 30 μm to 55 μm, preferably 30 μm to 50 μm.

Item 4: The method of any one of the preceding items, wherein the particle size is the median particle size of the cumulative volume distribution as determined according to ASTM E 1772-95 (Reapproved 2001) or ISO 13319-1:2021, in particular ASTM E 1772-95 (Reapproved 2001).

Item 5: The method of any one of the preceding items, wherein said agarose is crosslinked agarose.

Item 6: The method of item 5, wherein said agarose is 6% crosslinked agarose.

Item 7: The method of any one of the preceding items, wherein the HIC material has a ligand density from 5 μmol/mL to 60 μmol/mL, in particular 5 μmol/mL to 30 μmol/mL.

Item 8: The method of any one of the preceding items, wherein the HIC material has a ligand density from 10 μmol/mL to 60 μmol/mL, in particular 10 μmol/mL to 30 μmol/mL

Item 9: The method of any one of the preceding items, wherein the hydrophobic ligand is selected from the group consisting of aryl groups, alkyl groups and combinations thereof.

Item 10: The method of any one of the preceding items, wherein the hydrophobic ligand is selected from the group consisting of a butyl group, a phenyl group, an octyl group and combinations thereof.

Item 11: The method of item 10, wherein the hydrophobic ligand is a butyl group.

Item 12: The method of item 10, wherein the hydrophobic ligand is an octyl group.

Item 13: The method of item 10, wherein the hydrophobic ligand is a phenyl group.

Item 14: The method of any one of the preceding items, wherein the liquid applied to the HIC material comprises at least one salt, in particular at a concentration of at least 0.4M, preferably of at least 0.5 M.

Item 15: The method of item 14, wherein the concentration of the at least one salt, in particular a sulfate salt, in the liquid is less than 1M.

Item 16: The method of item 14 or 15, wherein the at least one salt comprises sulfate anions.

Item 17: The method of item 16, wherein the at least one salt is selected from sodium sulfate, potassium sulfate, lithium sulfate, ammonium sulfate and combinations thereof.

Item 18: The method of any one of the preceding items, wherein the liquid applied to the HIC material comprises 0.4M to 0.75M sodium sulfate.

Item 19: The method of any one of the preceding items, wherein the liquid applied to the HIC material has a pH from about 5 to about 8.

Item 20: The method of any one of the preceding items, further comprising (1) washing the HIC material having bound thereto said soluble complement receptor protein or a variant thereof, in particular wherein the wash buffer has a salt concentration of more than 0.1M, preferably more than 0.3M, and (2) eluting the soluble complement receptor protein or variant thereof from the HIC material by applying an elution buffer to the HIC material, said elution buffer having a salt concentration of less than 0.3M, preferably less than 0.1M.

Item 21: The method of item 20, wherein said eluting comprises applying a gradient with decreasing salt concentration.

Item 22: The method of any one of the preceding items, wherein the yield of the HIC step is more than 80%.

Item 23: The method of any one of the preceding items, wherein the host cell protein content in the HIC eluate is less than 1 μg/mg protein, preferably less than 0.5 μg/mg protein, more preferably less than 150 ng/mg protein.

Item 24: The method of any one of the preceding items, wherein the host cell protein depletion factor of the HIC is greater than 50.

Item 25: The method of any one of the preceding items, wherein the soluble complement receptor protein or a variant thereof is purified from bioreactor harvest, in particular cell free bioreactor harvest.

Item 26: The method of any one of the preceding items, wherein the soluble complement receptor protein or variant thereof comprises an amino acid sequence corresponding to the amino acid sequence of SEQ ID NO:2, in particular is a soluble complement receptor protein variant consisting of SEQ ID NO:2.

Item 27: The method of any one of the preceding items, wherein the soluble complement receptor protein or variant thereof is a sCR1 variant lacking long homologous repeat region LHR-D and/or is a sCR1 variant that does not comprise an amino acid sequence corresponding to amino acids 1393 to 1971 of SEQ ID NO:1.

Item 28: The method of any one of the preceding items, further comprising, prior to said HIC, the following steps
a. subjecting a mixture comprising said complement receptor protein or variant thereof to a cation exchange capture chromatography, in particular using NaCl,
b. subjecting the composition to an anion exchange chromatography, in particular using NaCl.

Item 29: The method of any one of the preceding items, wherein the soluble complement receptor protein or variant thereof is a recombinant protein, in particular a recombinant soluble complement receptor protein type 1 or a variant thereof.

### DESCRIPTION OF THE DRAWINGS

FIG. 1 depicts a chromatogram of a Phenyl Sepharose HP Chromatography conducted in accordance with Example 4, using sodium sulfate as a salt. The dotted line shows the conductivity trace, the solid line the UV-Signal.

FIG. 2 depicts a UV chromatogram of a Phenyl Sepharose HP Chromatography conducted in accordance with Example 4, using lithium sulfate as a salt. The dotted line shows the conductivity trace, the solid line the UV-Signal.

### DETAILED DESCRIPTION OF THE INVENTION

The present invention relates to a method for purifying a soluble complement receptor protein or a variant thereof, comprising subjecting a liquid comprising said complement receptor protein or variant thereof to a hydrophobic interaction chromatography (HIC), wherein said HIC comprises applying said liquid to a particulate HIC material comprising agarose and a hydrophobic ligand, wherein the particle size of said particulate HIC material is less than 60 μm.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

The term “and/or”, e.g. “X and/or Y” shall be understood to mean either “X and Y” or “X or Y” and shall be taken to provide explicit support for both meanings or for either meaning.

### Soluble Complement Receptor Proteins and Variants Thereof

Complement receptor type 1 (CR1), also known as C3b/C4b receptor or CD35, is a member of the family of regulators of complement activation. Naturally occurring CR1 is present on the membranes of erythrocytes, monocytes/macrophages, granulocytes, B cells, some T cells, splenic follicular dendritic cells, and glomerular podocytes, and mediates cellular binding to particles and immune complexes that have activated complement. Human CR1 has a 41 amino acid signal peptide, an extracellular domain of 1930 residues, a 25-residue transmembrane domain and a 43-amino acid C-terminal cytoplasmic region.

Soluble complement receptor type 1 (sCR1) is naturally produced by cleavage of cell surface CR1 and plays a role in the control of complement activation at sites of inflammation. It should be understood that reference to “sCR1” herein refers to soluble CR1, in particular a truncated CR1 which lacks the trans-membrane and cytoplasmic domains. For the purposes of nomenclature only and not limitation an exemplary sequence of human sCR1 including the signal peptide is set out in SEQ ID NO:1. Positions of amino acids are referred to herein by reference to sCR1 protein consisting of 1971 amino acids (e.g., as set out in SEQ ID NO:1). Full length sCR1 comprises four long homologous repeat (LHR) regions, i.e., LHR-A, B, C and D.

The sequence of sCR1 from other species can be determined using sequences provided herein and/or in publicly available databases and/or determined using standard techniques, e.g., as described in Ausubel el al, (editors), Current Protocols in Molecular Biology, Greene Pub. Associates and Wiley-Interscience (1988, including all updates until present) or Sambrook et al, Molecular Cloning: A Laboratory Manual, Cold Spring Harbor Laboratory Press (1989).

In some embodiments, the complement receptor protein or variant thereof used in the method of the invention is sCR1 or a variant thereof, in particular a sCR1 variant such as CSL040. In some embodiments, the complement receptor protein or variant thereof used in the method of the invention is human CR1 or a variant derived from human CR1, e.g. a variant comprising LHR-A, B and/or C of human CR1. In one embodiment the soluble complement receptor protein or variant thereof is a soluble form of CR1. An exemplary sequence of human CR1 is set out in UniProt entry no. P17927. In some embodiments, the complement receptor protein or variant thereof comprises SEQ ID No: 2.

As used herein the phrase “corresponding to” in reference to the position of amino acids in SEQ ID NO:1 should be understood as reference to amino acid residues or positions within a sCR1 sequence, and not necessarily a sequence comprising the entire SEQ ID NO: 1. For example, reference to “a position corresponding to amino acids 42 to 939 of SEQ ID NO:1” in a sCR1 sequence having a 41 amino acid N-terminal truncation (i.e., mature sCR1) would refer to amino acids at position 1 to 898 of the N-terminally truncated sequence. In one embodiment, the soluble complement receptor protein or variant thereof comprises or consists of amino acids 42-1971 of SEQ ID NO:1.

As used herein, the term “variant” refers to a sCR1, which has undergone substitution, deletion, addition and/or truncation of one or more amino acids. Variants include naturally occurring isoforms of soluble CR1. Variants also include a sCR1, which has undergone truncation and comprises SEQ ID NO: 2. One example of such a variant is CSL040. Further variants are described herein and in below cited documents.

The term “recombinant” shall be understood to mean the product of artificial genetic recombination. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

Various sCR1 variants are disclosed in WO 2019/218009 A1 and corresponding US 2021/238238 A (application Ser. No. 17/053,981). These documents and therein-disclosed variants are incorporated herein in their entirety by reference. In case of conflict with any incorporated documents, the contents of this specification shall prevail. Various sCR1 variants are also disclosed in Wymann et al. (2021) J Biol Chem. 296, see supra. In one embodiment, the sCR1 variant comprises or consists of amino acids 42 to 939 of SEQ ID NO:1;
amino acids 42 to 1392 of SEQ ID NO:1;
amino acids 42 to 1971 of SEQ ID NO:1;
amino acids 490 to 939 of SEQ ID NO:1;
amino acids 490 to 1392 of SEQ ID NO:1;
amino acids 490 to 1971 of SEQ ID NO:1; or
a corresponding sequence thereof.

In one embodiment, the sCR1 variant corresponds to SEQ ID NO:1, which is truncated at position 939 or 1392. In one embodiment, the sCR1 variant corresponds to SEQ ID NO: 1, which is truncated at position 42 or 490.

In one embodiment, the sCR1 variant of the present disclosure does not comprise an amino acid sequence corresponding to amino acids 1 to 41 of SEQ ID NO:1.

In one embodiment, the sCR1 variant of the present disclosure does not comprise an amino acid sequence corresponding to amino acids 940 to 1971 of SEQ ID NO:1.

In one embodiment, the sCR1 variant of the present disclosure does not comprise an amino acid sequence corresponding to amino acids 1393 to 1971 of SEQ ID NO:1.

In one embodiment, the sCR1 variant of the present disclosure does not comprise an amino acid sequence corresponding to amino acids 1 to 489 of SEQ ID NO:1.

In one embodiment, the sCR1 variant corresponds to amino acids 42 to 1392 of SEQ ID NO: 1. In one embodiment, the sCR1 variant comprises an amino acid sequence selected from the group consisting of: (i) an amino acid sequence corresponding to amino acids 42 to 939 of SEQ ID NO:1; and (ii) an amino acid sequence corresponding to amino acids 490 to 1392 of SEQ ID NO:1. sCR1 variants comprising residues 42 to 939 and/or residues 490 to 1392 of SEQ ID NO:1 have increased complement inhibitory activity.

In one embodiment, the sCR1 variant of the present disclosure comprises long homologous repeat (LHR) regions selected from the group consisting of: (i) LHR-A and LHR-B, but lacking LHR-C and LHR-D; (ii) LHR-A, LHR-B and LHR-C, but lacking LHR-D; (iii) LHR-B and LHR-C; C, but lacking LHR-A and LHR-D and (iv) LHR-B, LHR-C and LHR-D, but lacking LHR-A. In such embodiments, LHR region LHR-A comprises an amino acid sequence corresponding to amino acids 42 to 489 of SEQ ID NO: 1; LHR-B comprises an amino acid sequence corresponding to amino acids 490 to 939 of SEQ ID NO:1; LHR-C comprises an amino acid sequence corresponding to amino acids 940 to 1392 of SEQ ID NO:1; LHR-D comprises an amino acid sequence corresponding to amino acids 1393 to 1971 of SEQ ID NO:1. In one advantageous embodiment, the sCR1 variant of the present disclosure comprises long homologous repeat (LHR) regions LHR-A, LHR-B, LHR-C, but not LHR-D.

In one embodiment, the sCR1 variant is a recombinant protein. In one embodiment the sCR1 variant is a recombinant sCR1 variant comprising an amino acid sequence with at least 80%, at least 90%, at least 95, at least 99% sequence identity to SEQ ID: NO: 1, in particular an amino acid sequence having a length of at least 500, at least 600, at least 700, at least 800 or at least 900 amino acids.

In one embodiment, the sCR1 variant is monomeric (i.e., one copy of the sCR1 variant). In one embodiment, the sCR1 variant is dimeric, or dimerized (i.e., two copies of a sCR1 variant are linked in a fusion protein). In one embodiment, the sCR1 variant is multimeric, or multimerized (i.e., multiple copies of a sCR1 variant are linked in a fusion protein), see, e.g., WO 2019/218009 A1.

In one embodiment, the sCR1 variant for use in the present disclosure comprises at least two sialylated glycans (e.g., di-, tri- or tetra-sialylated glycans). For example, a composition for use in any method described herein comprises a sialylated sCR1 variant glycoform. In one example, a sialylated sCR1 variant glycoform for use in any method described herein comprises di-, tri- or tetra-sialylated glycoforms. Methods for producing variant sCR1 glycoforms comprising at least two sialylated glycans (e.g., di-, tri- or tetra-sialylated glycans), will be apparent to the skilled person and/or described herein. In some embodiments, at least 30% of the sialylated sCR1 variant glycoforms comprise mono-, di-, tri- and/or tetra-sialylated glycans.

Exemplary methods for determining the biological activity of the sCR1 variant of the disclosure will be apparent to the skilled person. For example, methods for determining inhibitory activity of the classical, lectin and/or alternative pathway are known in the art.

Exemplary compounds that can be conjugated to a sCR1 variant of the disclosure and methods for such conjugation are known in the art. In one example, the sCR1 variant of the present disclosure is conjugated to a half-life extending moiety. For example, the half-life extending moiety may be albumin, an antibody Fc region or functional fragments or variants thereof. sCR1 variants conjugated to half-life extending moieties such as human serum albumin (HSA) or IgG₄ Fc are known in the art, e.g. several examples are described in the above mentioned incorporated patent documents. In one embodiment, the sCR1 variant comprises a half-life extending moiety selected from the group consisting of a human serum albumin or functional fragment thereof, an immunoglobulin Fc region or functional fragment thereof, afamin, alphafetoprotein, vitamin D binding protein, antibody fragments that bind to albumin and polymers.

### HIC Material

The method of the invention comprises subjecting an, in particular aqueous, liquid comprising said complement receptor protein or variant thereof to a hydrophobic interaction chromatography (HIC), wherein said HIC comprises applying said liquid to a particulate (e.g. substantially spherical) HIC material comprising agarose and a hydrophobic ligand, wherein the particle size of said particulate HIC material is less than 60 μm.

As used herein, the term “particle size” refers to the median particle size of the cumulative volume distribution. It may be determined according to ASTM E 1772-95 (Reapproved 2001), “Standard Test Method for Particle Size Distribution of Chromatography Media by Electric Sensing Zone Technique” and/or according to ISO 13319-1:2021 “Determination of particle size distribution—Electrical sensing zone method—Part 1: Aperture/orifice tube method”. In some embodiments, particle size is determined according to ASTM E 1772-95. In some embodiments, particle size is determined according to ISO 13319-1:2021.

The HIC material comprises or substantially consists of agarose having covalently linked thereto hydrophobic groups. The hydrophobic group may be a hydrophobic hydrocarbon group. Preferably, the hydrophobic group is a hydrophobic alkyl group or a hydrophobic aryl group. More preferably, the hydrophobic group is a butyl group, a phenyl group, or an octyl group. Most preferably, the hydrophobic group is a phenyl group.

The HIC material is typically composed of particles. In some embodiments the particles are substantially spherical.

According to the invention the particle size of the particles is less than 60 μm. Preferably, the particle size is less than 55 μm, in particular less than 50 μm. In one embodiment, the particle size is 40 μm or less.

In some embodiments, the particle size is greater than 10 μm, in particular greater than 20 μm, preferably greater than 30 μm.

In other preferred embodiments the particle size is from about 10 μm to about 55 μm, or from about 20 μm to about 50 μm, or from about 25 μm to about 45 μm. Advantageous is also a particle size from about 30 μm to about 40 μm. In some further embodiments, the particle size is from about 20 μm to about 55 μm, in particular about 30 μm to about 55 μm, preferably about 30 μm to about 50 μm.

In some embodiments, the HIC material used in the method of the invention has a ligand density from about 10 μmol/mL to about 60 μmol/mL, in particular from about 10 μmol/mL to about 50 μmol/mL, preferably from about 10 μmol/mL to about 30 μmol/mL. In some embodiments, the HIC material used in the method of the invention has a ligand density from 20 μmol/mL to 40 μmol/mL. In some embodiments, the HIC material used in the method of the invention has a ligand density greater than 5 μmol/mL, in particular greater than 10 μmol/mL, preferably greater than 20 μmol/mL. In some embodiments, the HIC material used in the method of the invention has a ligand density lower than 60 μmol/mL, in particular lower than 50 μmol/mL. The skilled person is able to determine the ligand density using methods known in the art. In case of aromatic ligands, e.g. phenyl, the ligand density may be determined by measuring the absorbance of a hydrolyzed dried sample of the HIC material. The ligand density is then calculated from the molar absorptivity & for the aromatic ligand using Lambert Beer's law. In case of alkyl ligands, e.g. butyl or octyl, the ligand density may be determined by cleaving the ether linkages by boron tribromide and quantifying the bromoalkanes (e.g. bromobutanes or bromooctanes) that are formed by gas chromatography. When reference is made to ligand density using the unit “μmol/mL” this relates to the amount of ligand in μmol in relation to resin in mL.

In some embodiments, the HIC material used in the method of the invention has a ligand density from about 0.2 μmol/mg to about 0.8 μmol/mg, in particular from about 0.25 μmol/mg to about 0.6 μmol/mg, preferably from about 0.3 μmol/mg to about 0.4 μmol/mg. When reference is made to ligand density using the unit “μmol/mg” this relates to the amount of ligand in μmol in relation to resin in mg.

Suitable HIC materials for use in the method of the present invention include, but are not limited to, the following, all of which can be obtained from the company Cytiva®.
Capto® Phenyl ImpRes, having the following characteristics. Matrix: high-flow agarose; hydrophobic ligand: phenyl group; particle size: about 40 μm; ligand density: about 9 μmol/mL medium.
Phenyl Sepharose® High Performance, having the following characteristics. Matrix: 6% cross-linked agarose beads; hydrophobic ligand: phenyl group; particle size: about 34 μm; ligand density: about 25 μmol/mL medium.
Butyl Sepharose® High Performance, having the following characteristics. Matrix: 6% cross-linked agarose beads; hydrophobic ligand: butyl group; particle size: about 34 μm; ligand density: about 50 μmol/mL medium.

In certain embodiments the agarose is crosslinked agarose. Crosslinked agarose beads are usually available in 2%, 4% and 6% agarose content, while 6% crosslinked agarose is preferred.

### Salt Solution for Binding of the Protein to the HIC Column

Adsorption of the proteins to a HIC column is favoured by high salt concentrations, but the actual concentrations can vary over a wide range depending on the nature of the protein and the particular HIC ligand chosen. The liquid comprising the sCR1 or variant thereof can for example be loaded onto the HIC column by spiking the preceding eluate with solid salt or by in-line dilution with a high-salt buffer. Details on suitable salts and concentrations for binding the protein to the column are provided hereinafter.

Suitable salts that can be used include, but are not limited to, sodium chloride, magnesium chloride, sodium sulfate, ammonium sulfate, lithium sulfate and potassium sulfate. In some embodiments, a sulfate is used. Alkali sulfates or ammonium sulfate have shown to be advantageous, in particular ammonium sulfate, lithium sulfate or sodium sulfate. Sodium sulfate is particularly advantageous.

In some embodiments, a sulfate salt is used at a concentration that avoids protein precipitation, In some embodiments, a sulfate salt at a concentration of at least 0.4 M, preferably at least 0.5 M, is used, wherein the concentrations is low enough to avoid precipitation of the soluble complement receptor. Protein precipitation can, for example, be detected by turbidity measurements (see examples). Examples for suitable concentration ranges are provided hereinafter.

In some embodiments, a salt, in particular sulfate, concentration of 500-900 mM, e.g. about 700 mM in the liquid is used for applying the sCR1 or variant thereof onto the HIC material.

If ammonium sulfate is used, the concentration should be less than 2M. In some embodiments, suitable concentrations of ammonium sulfate in the loading buffer may be about 0.5M to about 1.5M, preferably about 0.5M to about 1M. Unfavourable ammonium sulfate concentrations were found to lead to negative effects on yield, e.g. in view of possible protein precipitation.

If sodium sulfate is used, the concentration preferably is less than 1M. In some embodiments, suitable concentrations of sodium sulfate in the loading buffer may be about 0.4M to about 0.8M, in particular about 0.5 M to about 0.75 M, preferably about 0.6M to about 0.75M, e.g. about 0.7 M. Unfavourable sodium sulfate concentrations were found to lead to negative effects on yield, e.g. in view of possible protein precipitation.

If lithium sulfate is used, the concentration preferably is less than 1M. In some embodiments, suitable concentrations of sodium sulfate in the loading buffer may be about 0.4M to about 1M, in particular about 0.6M to about 1M, preferably about 1M. Unfavourable lithium sulfate concentrations were found to lead to negative effects on yield, e.g. in view of possible protein precipitation.

If potassium sulfate is used, the concentration preferably is less than 0.7M. In some embodiments, suitable concentrations of sodium sulfate in the loading buffer may be about 0.3M to about 0.8M, in particular about 0.4 to about 0.6M, preferably about 0.5M. Unfavourable potassium sulfate concentrations were found to lead to negative effects on yield, e.g. in view of possible protein precipitation.

In some embodiments, salt concentrations of other salts may range from about 0.5M to about 2M.

Preferably the at least one salt is an inorganic salt. In some advantageous embodiments, only one inorganic salt is used or at least 90%, in particular at least 95%, by weight of the used inorganic salts used is attributed to one inorganic salt. In other embodiments, more than one salts may be used, in particular mixtures of inorganic salts described herein.

Preferably, a salt concentration is used below the protein saturation limit, i.e. a salt concentration below the concentration at which the soluble complement receptor protein precipitates, in particular a salt concentration that is below the concentration causing precipitation, but not more than 1M below the concentration causing precipitation, in particular not more than 0.5 M below the concentration causing precipitation. For example, if described soluble complement receptor precipitation occurs at a salt concentration of 1.2 M as determined by turbidity, a suitable concentration may be 0.8 M, which is below 1.2 M, but just 0.4 M below it (less than 1 M below it). It was found that relatively high salt concentrations, but not as high as to cause soluble complement receptor precipitation, lead to particular high yields, in particular for sulfate salts. Salt concentrations that are much lower or salt concentrations that are higher and cause protein precipitation were found to provide lesser yields.

After loading the sCR1 or variant thereof onto the HIC material, e.g. the HIC column, the HIC material may be washed using the high-salt buffer used as loading buffer. This removes protein contaminants and other impurities.

### Elution of the Protein

Elution, whether stepwise or in the form of a gradient, can be accomplished in a variety of ways: (a) by changing the salt concentration, (b) by changing the polarity of the solvent or (c) by adding detergents. By decreasing salt concentration adsorbed proteins are eluted in order of increasing hydrophobicity. In some embodiments, elution may be done by applying a linear salt gradient elution from said high salt concentration to a lower concentration such as 0 mM. Changes in polarity may be affected by additions of solvents such as ethylene glycol or (iso) propanol thereby decreasing the strength of the hydrophobic interactions. Detergents function as displacers of proteins and have been used primarily in connection with the purification of membrane proteins.

Preferably, the elution is performed by applying an, in particular aqueous, elution buffer to the HIC material having a lower salt concentration than the loading buffer. Often this is done by applying a gradient with decreasing salt concentration. For example, the sCR1 or variant thereof may be eluted by a linear gradient to 0 mM salt.

Preferably, the yield of the HIC step is at least 80%, in particular at least 85%, preferably at least 90%.

The host cell protein depletion factor is preferably at least 50, more preferably at least 60 or more.

### Further Purification Steps

The method of the invention may comprise additional purification steps (e.g. prior and/or after HIC), including cation exchange chromatography (capture chromatography), anion exchange chromatography, and filtration.

The mixture comprising the sCR1 or variant thereof may be subjected to ion exchange chromatography as a first step. Various anionic or cationic substituents may be attached to matrices in order to form anionic or cationic supports for the chromatography. Anionic exchange substituents include diethylaminoethyl (DEAE), quaternary aminoethyl (QAE) and quaternary amine (Q) groups. Cationic exchange substituents include carboxymethyl (CM), sulfoethyl (SE), sulfopropyl (SP), phosphate (P) and sulfonate(S). Cellulosic ion exchange resins such as DE23, DE32, DE52, CM-23, CM-32 and CM-52 are available from Whatman Ltd. Maidstone, Kent, U.K. SEPHADEX®-based and cross-linked ion exchangers are also known. For example, DEAE-, QAE-, CM-, and SP-SEPHADEX® and DEAE-, Q-, CM- and S-SEPHAROSE® are available from Pharmacia AB. Further both DEAE and CM derivatized ethylene glycol-methacrylate copolymer such as TOYOPEARL DEAE-650S and TOYOPEARL CM-650S are available from Tosoh Bioscience, King of Prussia, PA.

A sequence of purification steps may comprise the following, in particular in this order:
1. Harvest of cell culture supernatant and clarification
2. Capture cation exchange chromatography by applying a linear salt gradient of NaCl
3. Virus inactivation
4. Depth filtration
5. Anion exchange chromatography in flow through and/or bind elute mode using NaCl
6. Hydrophobic Interaction chromatography with Phenyl Sepharose HP in bind elute mode
7. Filtration steps

### Exemplary HIC Protocol

An exemplary protocol for the HIC purification of CSL040 is described in the following: Using sodium sulphate as chaotropic salt, it is possible to bind the CSL040 to the resin before the product can be eluted by reduction of high salt conditions using a linear gradient. The Phenyl Sepharose HP column is packed to a 15±1 cm bed height. The column capacity is 15-20 g CSL040/L resin. The Toyopearl™ NH2 750F eluate is spiked with solid sodium sulphate to a calculated concentration of 700 mM to achieve proper binding of CSL040 to the Phenyl Sepharose HP resin. The solid salt is added slowly under constant stirring at room temperature to a conductivity level of around 80 mS/cm. No pH adjustment is performed after addition of sodium sulphate. The feedstock is applied to the column with a targeted linear flow rate of 115 cm/h. After loading and postload-wash with HIC EQ-buffer (700 mM Sodium Sulfate, 148 mM Sodium Phosphate, pH 5.5±0.1), the target protein is eluted by linear gradient elution from 700 mM sodium sulphate to 0 mM in 10 CV. The collection started once the UV-signal exceeds 500 mAU/mm and stops when the signal drops below 150 mAU/mm. The eluate volume resembles approx. 4-5 CV. Residual HMWCs and CHO HCPs from Toyopearl NH2 750F eluate are further depleted (HMWC≤1% and CHO HCP level <100 ppm). HIC EQ-Buffer: 700 mM Sodium Sulfate, 148 mM Sodium Phosphate, pH 5.5. HIC Elution Buffer: 148 mM Sodium Phosphate, pH 5.5.

### EXAMPLES

### Example 1: Generation of CSL040 and Derivatives Thereof

CSL040 is a soluble variant of human CR1 comprising the LHR domains A, B and C. The amino acid sequence of mature CSL040 lacking the signal peptide is shown in SEQ ID NO:2, which is the polypeptide used in the present examples. CSL040 derivatives were generated and expressed as described in Example 1 of US 2021/238238 A (U.S. application Ser. No. 17/053,981, corresponds to WO 2019/218009 A1). CSL040 was generated accordingly. The disclosure of US 2021/238238 A is incorporated herein by reference in its entirety. The generation of CSL040 derivatives with 8× His tag is also described in Wyman et al. “A novel soluble complement receptor 1 fragment with enhanced therapeutic potential.” Journal of Biological Chemistry 296 (2021).

For purification, the cell free bioreactor harvest was pH and conductivity adjusted before being loaded onto a cation exchange chromatography to capture the target protein. CSL040 was eluted by applying a linear salt gradient of sodium chloride. The respective eluate was virus inactivated and incubated for at least 2-4 h at room temperature. After depth filtration the filtrate was applied onto an anion exchange chromatography resin. The target protein was eluted with an eluent containing sodium chloride.

The resulting partially purified CSL040 was used in the further experiments.

### Example 2: Effect of Salt

The effects of salt type and salt concentration on product quality was tested. To evaluate the influence of different chaotropic salts on the product related quality parameters (e.g. aggregate level), the CSL040 product quality was tested in the presence of different salt concentrations. The partially purified product was spiked with solid salt to obtain different salt concentrations (see table 1). The product related quality attributes were tested by analytical size-exclusion chromatography and percentages for high and low molecular weight components are provided. Furthermore, the turbidity (NTU-Nephelometric Turbidity Unit) of the samples was tested to evaluate the applicable salt concentration were protein salting-out effect becomes visible. It can clearly be seen in table 2 that applying sodium sulfate concentrations greater 0.75 M and ammonium sulfate concentrations greater 1 M led to higher turbidities indication protein precipitation. The results are shown in tables 1 and 2.

TABLE 1
Sample composition and analysis.

| | HMWC | Main | LMWC |
|---|---|---|---|
| Sample composition | (%) | (%) | (%) |
| 50 mM MES + 0.5M (NH₄)₂SO₄, pH 6.0 | 3.0 | 94.3 | 2.6 |
| 50 mM MES + 1M (NH₄)₂SO₄. pH 6.0 | 4.2 | 93.1 | 2.7 |
| 50 mM MES + 0.5M Na₂SO₄. pH 6.0 | 2.3 | 94.9 | 2.7 |
| 50 mM MES + 0.75M Na₂SO₄. pH 6.0 | 3.1 | 94.3 | 2.6 |
| 50 mM MES + 1M NaCl. pH 6.0 | 2.2 | 95.0 | 2.8 |
| 50 mM MES + 2M NaCl. pH 6.0 | 3.2 | 93.5 | 3.3 |
| 50 mM MES + 3M NaCl. pH 6.0 | 4.5 | 90.9 | 4.6 |
| 50 mM MES + 0.5M MgCl₂. pH 6.0 | 2.2 | 94.9 | 2.9 |
| 50 mM MES + 1M MgCl₂. pH 6.0 | 2.6 | 94.4 | 3.0 |
| 50 mM MES + 2M MgCl₂. pH 6.0 | 3.1 | 93.8 | 3.1 |
| 50 mM MES + 1M KCl. pH 6.0 | 2.1 | 94.9 | 3.0 |
| 50 mM MES + 2M KCl. pH 6.0 | 2.8 | 94.6 | 2.6 |
| 50 mM MES + 3M KCl. pH 6.0 | 3.8 | 90.9 | 5.3 |

TABLE 2
Turbidity testing for salt concentrations.

| | Salt | |
|---|---|---|
| | Conc. | Turbidity |
| Salt | [M] | [NTU] |
| (NH₄)₂SO₄ | 0.5 | 2.41 |
| | 1.0 | 7.83 |
| | 2.0 | 2591 |
| Na₂SO₄ | 0.5 | 3.18 |
| | 0.75 | 25.60 |
| | 1.0 | 5007 |
| NaCl | 1.0 | 1.80 |
| | 2.0 | 4.00 |
| | 3.0 | 5.01 |
| MgCl₂ | 0.5 | 1.48 |
| | 1.0 | 3.18 |
| | 2.0 | 9.56 |
| KCl | 0.5 | 1.69 |
| | 1.0 | 2.33 |
| | 2.0 | 2.46 |

### Example 3: High Throughput Resin Screening

In a high throughput setup using TECAN system and Cytiva PreDictor Plates and Tosoh Seeker Plates, protein yield and host protein content (CHO HCP) were determined for different HIC resin materials. The following materials were compared.

TABLE 3
HIC resin materials.

| | | | Bead | Ligand |
|---|---|---|---|---|
| | | | Size | Density |
| | Vendor | Matrix | (μm) | (μmol/mL) |
| Phenyl Sepharose | Cytiva | Cross-linked agarose, | 34 | 25 |
| HP | | spherical | | |
| Phenyl FF (LS) | Cytiva | Highly cross-linked | 90 | 25 |
| | | agarose (6%) | | |
| Butyl Sepharose | Cytiva | Highly cross-linked | 90 | 40 |
| 4 FF | | agarose (4%) | | |
| Butyl-S | Cytiva | Highly cross-linked | 90 | 10 |
| Sepharose 6 FF | | agarose (6%) | | |
| Capto Phenyl HS | Cytiva | Highly cross-linked | 75 | 27 |
| | | agarose | | |
| Capto Phenyl | Cytiva | Highly cross-linked | 40 | 9 |
| Impres | | agarose, spherical | | |
| Capto Butyl | Cytiva | High Flow Agarose | 40 | n.a. |
| Impres | | | | |
| TOYOPEARL | Tosoh | polymethacrylate | 100 | n.a. |
| Hexyl-650C | | | | |
| TOYOPEARL | Tosoh | polymethacrylate | 65 | n.a. |
| Butyl-650M | | | | |
| TOYOPEARL | Tosoh | polymethacrylate | 65 | n.a. |
| Butyl-600M | | | | |
| TOYOPEARL | Tosoh | polymethacrylate | 100 | n.a. |
| SuperButyl-550C | | | | |
| TOYOPEARL | Tosoh | polymethacrylate | 65 | n.a. |
| Phenyl-650M | | | | |
| TOYOPEARL | Tosoh | polymethacrylate | 65 | n.a. |
| Phenyl-600M | | | | |
| TOYOPEARL | Tosoh | polymethacrylate | 65 | n.a. |
| PPG-600M | | | | |
| TOYOPEARL | Tosoh | polymethacrylate | 65 | n.a. |
| Ether-650M | | | | |

n.a.-not available
Impres-Improves Resolution
HS-High Substitution
LS-Low Substitution
FF-Fast Flow

The resins to be tested were transferred into 96-well plates, containing between 20 μL to 50 μL of each tested resin. 2 load densities (20 mg/mL and 40 mg/mL) were tested to evaluate the binding capacity of each resin, the purity of the target protein and the residual impurity level (e.g. CHO HCP) to identify suitable HIC resins for CSL040 purification. In brief the method for high-throughput resin screening. Each well was equilibrated with an equivalent of 5 volumes of resin using an equilibration buffer containing the same salt concentration as in the tested load material. After application of the respective salt spiked load material the resins were washed with an equivalent of 5 resin volumes with high salt equilibration buffer. Elution of the target protein was achieved by lowering the salt concentration by applying 2 step elutions. The first step halves the salt concentration, the second one reduces the salt concentration to 0 mM. The respective eluates were analysed for product content by UV measurement (yield) and CHO HCP content. The yields are shown in table 4 below.

TABLE 4
High-Throughput resin screening of Cytiva and Tosoh resins using
Cytiva PreDictor Plates and Tosoh Seeker Plates, respectively.

| | Yield (%) | | | |
|---|---|---|---|---|
| Resin | Na₂SO₄ | | (NH₄)₂SO₄ | |
| Salt concentration (M) | 0.45 | | 1.0 | |
| Resin Load (mg/mL) | 20 | 40 | 20 | 40 |
| Phenyl Sepharose High | 68.4 | 44.4 | 78.0 | 56.1 |
| Performance | | | | |
| Butyl Sepharose High | 71.2 | 51.4 | 80.8 | 61.4 |
| Performance | | | | |
| Capto Phenyl ImpRes | 68.7 | 42.4 | 73.0 | 46.2 |
| Capto Butyl ImpRes | 60.2 | 44.6 | 78.3 | 53.2 |
| TOYOPEARL Hexyl- | 10.6 | 2.9 | 15.3 | 3.7 |
| 650C | | | | |
| TOYOPEARL Butyl- | 36.4 | 24.1 | 32.8 | 24.2 |
| 650M | | | | |
| TOYOPEARL Butyl- | 51.6 | 22.9 | 47.0 | 22.0 |
| 600M | | | | |
| TOYOPEARL | 26.1 | 9.9 | 22.0 | 9.3 |
| SuperButyl-550C | | | | |
| TOYOPEARL Phenyl- | 39.9 | 25.7 | 40.5 | 23.5 |
| 650M | | | | |
| TOYOPEARL Phenyl- | 30.0 | 19.1 | 46.8 | 17.8 |
| 600M | | | | |
| TOYOPEARL PPG- | 39.5 | 22.7 | 49.7 | 21.3 |
| 600M | | | | |
| TOYOPEARL Ether- | 4.1 | 2.2 | 6.1 | 3.6 |
| 650M | | | | |

### Example 4: Column Format

To confirm the high-throughput screening results, these were repeated at a 1 mL column scale on selected resins using sodium sulfate. Therefore, partially purified CSL040 was spike with the respective amount of solid sodium sulfate to a concentration of 0.7M. After loading to a target concentration of 20 mg CSL040 per mL resin the column was washed with equilibration buffer containing an equal concentration of sodium sulfate before eluting CSL040 from the resin using a linear gradient over 10 column volumes to final sodium sulfate concentration of 0M. The respective yield was calculated based on the UV-measured protein concentration in the eluate pool. Further experiments were carried out using different salts and/or salt concentrations. The results are summarized in Table 5.

TABLE 5
Column resins, conditions and yields.

| Resin | Condition | Yield |
|---|---|---|
| Phenyl Sepharose HP | Up to 3M MgCl₂ | No |
| | | binding |
| Phenyl Sepharose HP | 700 mM Na₂SO₄, pH 7.0 | 86% |
| | 700 mM Na₂SO₄, pH 5.5 | 80% |
| Capto Phenyl HS | 700 mM Na₂SO₄, pH 7.0 | 41% |
| | 700 mM Na₂SO₄, pH 5.5 | 38% |
| Phenyl Sepharose HP | 1M Na₂SO₄, pH 7.0 | 83% |
| Phenyl Sepharose FF (LS) | | 46% |
| Butyl-S FF | | 26% |
| Butyl-FF | | 53% |
| Phenyl Sepharose HP | 3M NaCl, pH 7.0 | 61% |
| Phenyl Sepharose FF (LS) | | 30% |
| Butyl-S FF | | No |
| | | binding |
| Butyl-FF | | 43% |
| Capto Phenyl ImpRes | 700 mM Na₂SO₄, pH 5.5 | 72% |
| Capto Butyl ImpRes | | 57% |

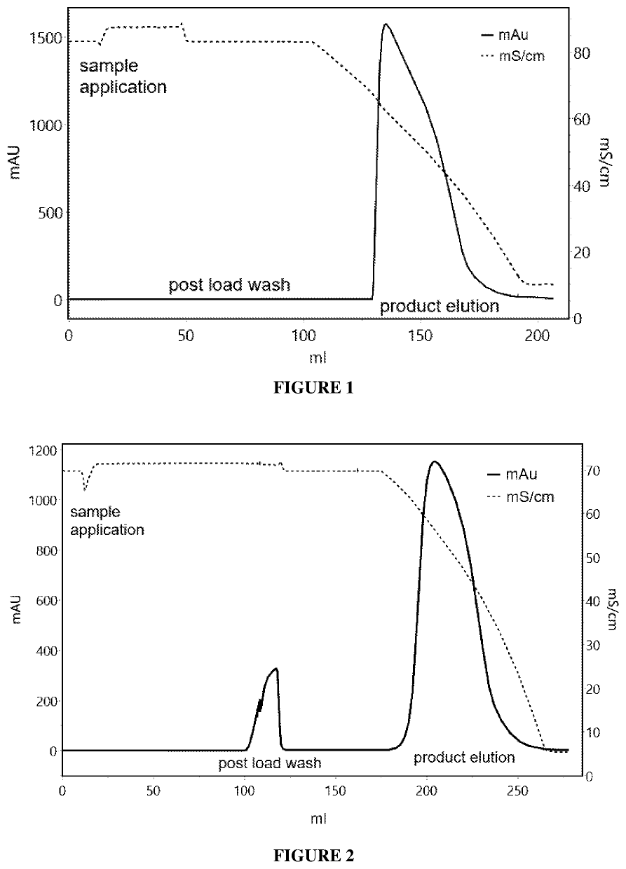

An exemplary chromatogram of a Phenyl Sepharose HP Chromatography using sodium sulfate as a salt is shown in FIG. 1. An exemplary chromatogram of a Phenyl Sepharose HP Chromatography using lithium sulfate as a salt is shown in FIG. 2. Potassium sulfate was also successfully tested as a salt in a Phenyl Sepharose HP Chromatography.

1. A method for purifying a soluble complement receptor protein or a variant thereof, comprising subjecting a liquid comprising said complement receptor protein or variant thereof to a hydrophobic interaction chromatography (HIC), wherein said HIC comprises applying said liquid to a particulate HIC material comprising agarose and a hydrophobic ligand, wherein the particle size of said particulate HIC material is less than 60 μm.

2. The method of claim 1, wherein said agarose is crosslinked agarose.

3. The method of claim 1, wherein the particle size is from 30 μm to 50 μm.

4. The method of claim 1, wherein the HIC material has a ligand density from 5 μmol/mL to 60 μmol/mL.

5. The method of claim 1, wherein the hydrophobic ligand is selected from the group consisting of aryl groups, alkyl groups, and combinations thereof.

6. The method of claim 1, wherein the liquid applied to the HIC material comprises at least one salt at a concentration of at least 0.4M.

7. The method of claim 6, wherein the concentration of the at least one salt is below the concentration at which the soluble complement receptor protein precipitates.

8. The method of claim 6, wherein the at least one salt comprises a sulfate anion.

9. The method of claim 8, wherein the at least one salt is selected from sodium sulfate, potassium sulfate, lithium sulfate, ammonium sulfate, and combinations thereof.

10. The method of claim 1, wherein the liquid applied to the HIC material has a pH from about 5 to about 8.

11. The method of claim 1, further comprising (1) washing the HIC material having bound thereto said soluble complement receptor protein or a variant thereof, and (2) eluting the soluble complement receptor protein or a variant thereof from the HIC material by applying an elution buffer to the HIC material, said elution buffer having a salt concentration of less than 0.3M.

12. The method of claim 1, wherein the host cell protein content in the HIC eluate is less than 1 μg/mg protein.

13. The method of claim 1, wherein the host cell depletion factor of the HIC is greater than 50.

14. The method of claim 1, wherein the soluble complement receptor protein or variant thereof is a recombinant protein, in particular a recombinant soluble complement receptor protein-type 1 or variant thereof.

15. The method of claim 1, further comprising, prior to said HIC,
(i) subjecting a mixture comprising said complement receptor protein or variant thereof to a cation exchange capture chromatography, and
(ii) subjecting the composition to an anion exchange chromatography.

16. The method of claim 4, wherein the HIC material has a ligand density from 10 μmol/mL to 30 μmol/mL.

17. The method of claim 9, wherein the at least one salt is selected from sodium sulfate, lithium sulfate, ammonium sulfate and combinations thereof.

18. The method of claim 14, wherein the soluble complement receptor protein or variant thereof is a recombinant soluble complement receptor protein type 1 or variant thereof.

19. The method of claim 1, wherein the host cell protein content in the HIC eluate is less than 1 μg/mg protein or the host cell protein depletion factor of the HIC is greater than 50 or both.

20. The method of claim 1, further comprising, prior to said HIC,
(i) subjecting a mixture comprising said complement receptor protein or variant thereof to a cation exchange capture chromatography, and
(ii) subjecting the composition to an anion exchange chromatography.
